(19) United States
(12) Patent Application Publication
(10) Pub. No.: US 2026/0085316 A1
(43) Pub. Date: Mar. 26, 2026

(54) USE OF HOMEOBOX CONTAINING 1 (HMBOX1) INHIBITOR IN PREPARATION OF DRUG FOR PREVENTION AND/OR TREATMENT OF MUSCLE ATROPHY

(71) Applicant: Shanghai University, Shanghai (CN)

(72) Inventors: Junjie XIAO, Shanghai (CN); Jin LI, Shanghai (CN); Tingting YANG, Shanghai (CN); Yuying CHEN, Shanghai (CN); Yuwei YAN, Shanghai (CN)

(73) Assignee: Shanghai University, Shanghai (CN)

(21) Appl. No.: 19/340,973

(22) Filed: Sep. 26, 2025

(30) Foreign Application Priority Data
Sep. 26, 2024 (CN) 202411360353.2

(51) Int. Cl. C12N 15/113; A61P 21/00

(52) U.S. Cl. CPC C12N 15/113; A61P 21/00; C12N 2310/14; C12N 2310/531

(57) ABSTRACT

Use of a homeobox containing 1 (HMBOX1) inhibitor in preparation of a drug for prevention and/or treatment of muscle atrophy. Functional experiments at the cellular level show that inhibiting the expression of HMBOX1 may effectively suppress the occurrence of muscle atrophy. Furthermore, functional experiments at the animal level demonstrate that inhibiting HMBOX1 expression has preventive and therapeutic effects against muscle atrophy. Therefore, the HMBOX1 inhibitor is used for the prevention and/or treatment of muscle atrophy, offering a new approach for the development of drugs that inhibit muscle atrophy.

### CROSS REFERENCE TO THE RELATED APPLICATIONS

This application is based upon and claims priority to Chinese Patent Application No. 202411360353.2, filed on Sep. 26, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0298_Sequence_Listing.xml, created on 09/26/2025, and is 6,561 bytes in size.

### TECHNICAL FIELD

The present disclosure belongs to the technical field of biomedicine, and in particular relates to use of a homeobox-containing 1 (HMBOX1) inhibitor in preparation of a drug for prevention and/or treatment of muscle atrophy.

### BACKGROUND

Skeletal muscle, as the largest organ in the human body, plays a crucial role in vital physiological processes such as movement, metabolism, and respiration. Muscle atrophy is a group of degenerative diseases characterized by the loss of muscle fibers and the depletion of muscle cell proteins. Conditions such as heart failure, kidney failure, and other systemic diseases, as well as acute physical injuries like severe burns and major trauma, and diseases such as cancer, can all lead to the occurrence of muscle atrophy. Muscle atrophy severely impacts the quality of life of patients, increases the incidence of related diseases, and significantly affects normal living, placing a heavy burden on families and society. It has become one of the major diseases threatening the health of the population.

Currently, a considerable amount of clinical work has been conducted on the treatment of muscle atrophy, which mainly includes physical therapy, surgical interventions, gene therapy, nutritional supplementation, and certain broad-spectrum pharmacological treatments. Although these therapeutic approaches have achieved varying degrees of success, most treatments aim to slow down the progression of muscle atrophy rather than providing targeted interventions. This indicates that the current clinical treatment methods and their effectiveness are limited. Therefore, exploring the mechanisms underlying muscle atrophy and identifying early diagnostic indicators and intervention targets at the molecular level hold significant clinical importance.

Homeobox containing 1 (HMBOX1) is a member of the homeobox gene family, possessing a homeobox domain at its N-terminus and a DNA-binding domain at its C-terminus. It is a potential transcription factor that is highly conserved across different species. HMBOX1 was first discovered in the pancreas and is also expressed at high levels in skeletal muscle. It has been reported to participate in processes such as cell differentiation, autophagy, and apoptosis. However, there are limited reports on the function of HMBOX1 in skeletal muscle. HMBOX1 is a key regulatory factor that modulates the differentiation of bone marrow stromal cells (BMSCs), promoting their differentiation into endothelial cells (ECs). However, there are currently no other reports regarding the diagnosis and treatment of muscle atrophy involving HMBOX1.

### SUMMARY

In light of this, the present disclosure aims to provide use of an HMBOX1 inhibitor in preparation of a drug for prevention and/or treatment of muscle atrophy.

To achieve the above objective, the present disclosure provides the following technical schemes.

The present disclosure provides use of an HMBOX1 inhibitor in preparation of a drug for prevention and/or treatment of muscle atrophy.

In some embodiments, the muscle atrophy includes one or more of neurogenic muscle atrophy, disuse muscle atrophy, and steroid-induced muscle atrophy.

In some embodiments, the HMBOX1 inhibitor includes one or more of a regulator that reduces HMBOX1 expression, a protease or a nuclease that degrades an HMBOX1 product, and a regulator that decreases the HMBOX1 product.

In some embodiments, the regulator that reduces HMBOX1 expression includes a small interfering RNA (siRNA), a short hairpin RNA (shRNA), or a mircroRNA (miRNA).

In some embodiments, the regulator that reduces the HMBOX1 product includes an HMBOX1 antibody.

In some embodiments, the siRNA has the nucleotide sequence of SEQ ID NO: 1; and the shRNA has the target sequence of SEQ ID NO: 1.

In some embodiments, the drug slows down a reduction in muscle weight and a shrinkage in muscle fibers.

In some embodiments, the drug significantly inhibits an increase in muscle atrophy-specific ubiquitin ligase.

The present disclosure provides a drug for prevention and/or treatment of muscle atrophy, where the drug includes an active ingredient and a pharmaceutically acceptable carrier, and the active ingredient is the shRNA.

In some embodiments, the muscle atrophy includes one or more of neurogenic muscle atrophy, disuse muscle atrophy, and steroid-induced muscle atrophy.

Compared to the prior art, embodiments of the present disclosure have the following beneficial effects.

The present disclosure provides use of an HMBOX1 inhibitor in preparation of a drug for prevention and/or treatment of muscle atrophy. Functional experiments at the cellular level have demonstrated that inhibiting the expression of HMBOX1 may effectively suppress the occurrence of muscle atrophy. Furthermore, functional experiments at the animal level have shown that inhibiting HMBOX1 expression has a preventive and therapeutic effect on muscle atrophy. Therefore, in the present disclosure, the HMBOX1 inhibitor is utilized for the prevention and/or treatment of muscle atrophy, providing a new approach for the development of drugs that inhibit muscle atrophy.

### BRIEF DESCRIPTION OF THE DRAWINGS

FIG. 1 shows a plasmid map of pLKO.1 puro.

FIG. 2 shows results of the effect of Sh-HMBOX1 on the expression of HMBOX1.

FIGS. 3A-3D illustrate effects of different treatments on dexamethasone (Dex)-induced muscle atrophy at the cellular level. FIGS. 3A-3B show effects of different treatments on the diameter of myotubes induced by dexamethasone (Dex), with a scale bar of 100 μm, and FIGS. 3C-3D show effects of different treatments on the expression of muscle atrophy-specific ubiquitin ligases Atrogin-1 and MuRF-1 induced by dexamethasone (Dex); **p<0.01.

FIG. 4 illustrates effects of AAV8-sh-HMBOX1 on the expression of HMBOX1.

FIGS. 5A-5G illustrate results of the effects of different treatments on denervation (Den)-induced muscle atrophy. FIGS. 5A-5C show muscle weight changes in different treatment groups following Den-induced muscle atrophy, with muscle images displayed from left to right as follows: AAV8-sh-control+Sham, AAV8-sh-control+Den, AAV8-sh-HMBOX1+Sham, and AAV8-sh-HMBOX1+Den, with a scale bar of 1 cm. FIGS. 5D-5E show changes in muscle fibers in different treatment groups following Den-induced muscle atrophy. FIGS. 5F-5G show effects of different treatments on the muscle atrophy-specific ubiquitin ligases Atrogin-1 and MuRF-1 induced by Den; **p<0.01.

FIGS. 6A-6G illustrate effects of different treatment groups on immobilization (Imo)-induced muscle atrophy. FIGS. 6A-6C show impact of different treatment groups on muscle weight following Imo-induced muscle atrophy, with muscle images displayed from left to right as follows: Sham+AAV8-sh-control, Imo+AAV8-sh-control, Sham+AAV8-sh-HMBOX1, and Imo+AAV8-sh-HMBOX1, with a scale bar of 1 cm. FIGS. 6D-6E show changes in muscle fibers in different treatment groups following Imo-induced muscle atrophy. FIGS. 6F-6G show effects of different treatments on the muscle atrophy-specific ubiquitin ligases Atrogin-1 and MuRF-1 induced by Imo; *p<0.05, **p<0.01.

### DETAILED DESCRIPTION OF THE EMBODIMENTS

The present disclosure provides use of an HMBOX1 inhibitor in preparation of a drug for prevention and/or treatment of muscle atrophy.

In the present disclosure, HMBOX1 is a protein that plays a regulatory role in muscle atrophy. The coding sequence (CDS) of HMBOX1 encodes 420 amino acids, and the sequence of the HMBOX1 CDS region is as follows:

(SEQ ID NO: 4)
ATGCTCAGCTCCTTTCCAGTGGTTTTGCTGGAAACCATGTCTCACTACAC
AGATGAACCCAGATTTACCATAGAACAGATAGACCTGCTCCAGCGTCTTC
GGCGTACCGGGATGACCAAACATGAAATCCTTCATGCATTAGAAACTTTG
GACCGTCTTGATCAAGAGCATAGTGATAAATTTGGAAGGAGGTCCAGCTA
CGGGGGAAGCTCATATGGGAACAGTACCAACAACGTTCCAGCATCTTCCT
CTACAGCCACAGCTTCCACGCAGACCCAGCACTCGGGAATGTCCCCATCA
CCCAGCAACAGTTACGATACCTCCCCACTGCCTTGCACTACCAATCAAAA
TGGGAGGGAGAACAATGATCGATTGTCCACATCCAATGGGAAGATGTCAC
CATCTCGCTACCATGCAAACAGCATGGGTCAGAGGTCATATAGCTTTGAG
GCCTCAGAAGAGGACCTAGATGTAGATGATAAAGTGGAGGAATTAATGAG
GAGGGACAGCAGTGTGATAAAAGAGGAAATCAAAGCCTTTCTTGCCAATC
GGAGGATTTCCCAAGCAGTTGTTGCACAGGTAACAGGAATCAGTCAGAGT
CGAATCTCTCACTGGCTGCTGCAGCAGGGATCAGATCTGAGTGAGCAGAA
GAAAAGGGCGTTCTACCGATGGTATCAACTTGAGAAGACAAACCCTGGGG
CTACGCTAAGTATGAGACCTGCCCCCATTCCAATAGAGGACCCTGAATGG
AGACAAACACCTCCCCCAGTCTCCGCCACACCTGGAACCTTCCGGCTTCG
ACGAGGGAGTAGATTTACCTGGAGAAAGGAGTGCCTAGCTGTCATGGAAA
GTTACTTCAATGAGAACCAGTACCCAGATGAAGCAAAGAGAGAAGAAATT
GCCAATGCTTGCAATGCAGTCATACAGAAGCCAGGCAAAAAGCTGTCTGA
CCTGGAACGAGTTACCTCTCTGAAAGTATATAATTGGTTTGCTAATCGAC
GGAAGGAGATCAAGAGAAGAGCCAATATCGAAGCAGCAATCCTGGAGAGT
CATGGGATAGATGTACAGAGTCCAGGAGGCCATTCCAACAGTGACGATGT
GGACGGGAACGACTACTCCGAGCAGGATGACAGCACCAGCCATAGTGACC
ACCAAGATCCCATCTCGCTAGCTGTGGAGATGGCGGCCGTCAACCACACT
ATCTTGGCATTGGCCCGGCAGGGAGCCAATGAAATCAAGACAGAGGCCCT
GGATGATGACTGA.

The muscle atrophy mentioned herein preferably includes one or more of the following types: neurogenic muscle atrophy, disuse muscle atrophy, and steroid-induced muscle atrophy. Steroid-induced muscle atrophy, such as that induced by dexamethasone (Dex), is caused by the administration of glucocorticoids. Prolonged injection of dexamethasone can lead to a decrease in muscle mass and a reduction in muscle strength. Neurogenic muscle atrophy may occur in models induced by denervation (Den), which are commonly used to study muscle atrophy resulting from neuromuscular junction dysfunction. Disuse muscle atrophy refers to muscle atrophy resulting from a lack of physical activity or reduced mobility. The immobilization (Imo) model of muscle atrophy is designed to simulate conditions such as prolonged bed rest, lack of exercise, or limited mobility in humans, such as in patients who are bedridden for extended periods or astronauts in space.

In the present disclosure, the inhibitor can suppress the expression of HMBOX1 in cells and tissues, disrupt the stability of HMBOX1 in cells and tissues, reduce the activity of HMBOX1 in cells and tissues, or decrease the effective action time of HMBOX1 in cells or tissues. The HMBOX1 inhibitor preferably includes one or more of the following: a regulator that reduces HMBOX1 expression, a protease or a nuclease that degrades an HMBOX1 product, and a regulator that decreases the HMBOX1 product. The regulator that reduces HMBOX1 expression preferably includes a siRNA, a shRNA, or a miRNA. The regulator that decreases the HMBOX1 product preferably includes an HMBOX1 antibody. The siRNA preferably has the nucleotide sequence of SEQ ID NO: 1; and the shRNA preferably has the target sequence of SEQ ID NO: 1.

In the present disclosure, the drug is designed to slow down a reduction in muscle weight and a shrinkage of muscle fibers, while also significantly inhibiting the increase of muscle atrophy-specific ubiquitin ligase.

The present disclosure provides a drug for prevention and/or treatment of muscle atrophy, including an active ingredient and a pharmaceutically acceptable carrier, where the active ingredient is the aforementioned shRNA.

In the drug for the prevention and/or treatment of muscle atrophy according to the present disclosure, the muscle atrophy includes one or more of neurogenic muscle atrophy, disuse muscle atrophy, and steroid-induced muscle atrophy. The pharmaceutically acceptable carrier includes, but are not limited to, a diluent, a buffer, a suspending agent, an emulsion, a granule, an encapsulating agent, an excipient, a filler, a binder, an aerosol, a transdermal absorption agent, a wetting agent, a disintegrant, an absorption enhancer, a surfactant, a colorant, a flavoring agent, or an adsorbent carrier. The ultimate therapeutic effect of the drug is to inhibit the expression level of HMBOX1 in cells or tissues.

The following detailed description of the technical solutions provided by the present disclosure is based on specific examples, but these should not be construed as limiting the protection scope of the present disclosure.

### Example 1

1. Induction of Muscle Atrophy by Dex after Intervention with Sh-HMBOX1 Lentivirus in Cells

### 1.1 Construction of Sh-HMBOX1 Plasmid:

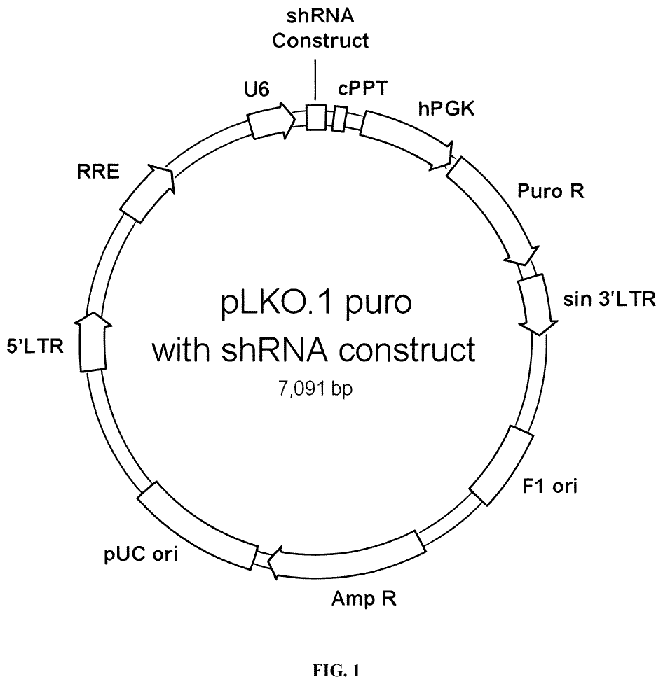

(S1) Vector: pLKO.1 puro. The plasmid map of pLKO.1 puro is shown in FIG. 1.
(S2) The shRNA sequence was designed based on the HMBOX1 siRNA sequence as follows. The nucleotide sequence of the siRNA is: GCTGGAAACCATGTCTCACTA (SEQ ID NO: 1):

Upstream primer: AgeI restriction enzyme
cohesive end + target sequence + loop +
anti-target sequence + TTTTTG,
(SEQ ID NO: 2)
5′-CCGGGCTGGAAACCATGTCTCACTACTCGAGTAGTGAGACATGGTTT
CCAGCTTTTTG-3′;
Downstream primer: EcoRI restriction enzyme
cohesive end + AAAAA + target sequence +
loop + anti-target sequence,
(SEQ ID NO: 3)
5′-AATTCAAAAAGCTGGAAACCATGTCTCACTACTCGAGTAGTGAGACA
TGGTTTCCAGC-3′;

(S3) The obtained shRNA sequence was sent to a company for synthesis.
(S4) The shRNA sequence was annealed.

TABLE 1
Annealing system for the shRNA sequence
System

| | |
|---|---|
| Upstream primer | 5 μL |
| Downstream primer | 5 μL |
| 10 × NEB buffer 2 | 5 μL |
| ddH₂O | 35 μL |

According to the annealing system of the shRNA sequence in Table 1, the mixture was heated to 95° C. for 4 minutes and then allowed to cool naturally to room temperature to obtain the annealed product.
(S5) Double enzyme digestion

TABLE 2
Double enzyme digestion system
Double enzyme digestion system

| | | |
|---|---|---|
| AgeI enzyme | 1.5 | μL |
| EcoRI enzyme | 1.5 | μL |
| Annealed product | 5 | μL |
| 10 × Buffer | 3 | μL |
| ddH₂O | 19 | μL |

Materials were added according to the double enzyme digestion system in Table 2, and incubated at 37° C. overnight to obtain the digestion product.
(S6) ligation.

TABLE 3
Ligation system
Ligation system

| | | |
|---|---|---|
| Digestion product | 50 | ng |
| pLKO.1 puro vector | 100 | ng |
| 10 × T4 Buffer | 2 | μL |
| T4 ligase | 2 | μL |

| | |
|---|---|
| ddH₂O | Up to 30 μL |

Materials were added according to the ligation system of table 3, and incubated at 16° C. overnight to obtain the Sh-HMBOX1 plasmid.
(S7) Transformation
a. Five milliliters of Sh-HMBOX1 plasmid and 50 μL of competent Escherichia. coli (Stbl3) were mixed and placed on ice for 20 minutes.
b. Heat shock was performed at 37° C. for 5 minutes or at 42° C. for 45 seconds.
c. One milliliter of liquid LB (without antibiotics) was added and incubation was conducted at 37° C. with shaking at 200-250 rpm for 1 hour.
d. After 1 hour, centrifugation was conducted at 3000 rpm for 5 minutes.
e. A resulting supernatant was discarded and the remaining 100-200 μL of competent cells were resuspended, and then spread onto Amp+ resistant LB plates.
f. Incubation was conducted at 37° C. overnight.
(S8) Single colonies was picked and subjected to shaking culture. The culture was used as a template to amplify the target gene, which was then subjected to gel electrophoresis for validation, and the strains with correct bands was selected for sequencing.

### 1.2 Packaging Sh-HMBOX1 Lentivirus:

(1) A 1.5 mL centrifuge tube was taken and a calcium-DNA mixture was prepared. The reagents used are listed in Table 4 (using an 8 cm cell culture dish as an example).

TABLE 4
Reagents for Packaging Sh-HMBOX1 Lentivirus

| agent | amount | |
|---|---|---|
| pMD2.G | 1.25 | μg |
| psPAX2 | 3.75 | μg |
| Sh-HMBOX1 plasmid | 5 | μg |
| CaCl₂ (2.5 mol/L) | 50 | μL |

| | |
|---|---|
| Distilled water | Made up to500 μL |

(2) A 2.0 mL centrifuge tube was taken, and 500 μL of 2×HEPES (N-2-hydroxyethylpiperazine-N-ethane-sulphonicacid) was added.
(3) The 2.0 mL centrifuge tube containing 500 μL of 2×HEPES was placed on a vortex mixer, and the calcium-DNA mixture was added dropwise while the liquid in the centrifuge tube was vortexing. This process took approximately 1 minute. After all the drops were added, the mixture was vortexed a few more times and allowed to stand for 20 minutes.
(4) During this time, fresh pre-warmed culture medium (Dulbecco's modification of Eagle's medium (DMEM)+10% fetal bovine serum (FBS)+1% penicillin-streptomycin) at 37° C. was exchanged for the 293T cells. The calcium phosphate-DNA precipitate was vigorously pipetted up and down about 8 times, and then the calcium phosphate-DNA suspension was immediately added dropwise to the cell culture medium of the aforementioned cells. The medium was gently mixed until it turned orange. Under a microscope, various sizes of precipitate were observed in the cell dish.
(5) The cells were incubated in a 37° C., 5% CO₂ humidified incubator.
(6) After 24 hours, the old culture medium was discarded, and 10 mL of new pre-warmed culture medium (DMEM+10% FBS) at 37° C. was added for continued culture.
(7) After 48 hours, the culture medium containing the lentivirus was collected and temporarily stored in a 4° C. refrigerator, and 10 mL of new pre-warmed culture medium (DMEM+10% FBS) at 37° C. was added for continued culture.
(8) After 72 hours, the culture medium containing the lentivirus was collected.
(9) The culture medium containing the lentivirus was filtered using a 0.45 μm filter, aliquoted, and stored at −80° C., resulting in the packaging of Sh-HMBOX1 lentivirus.

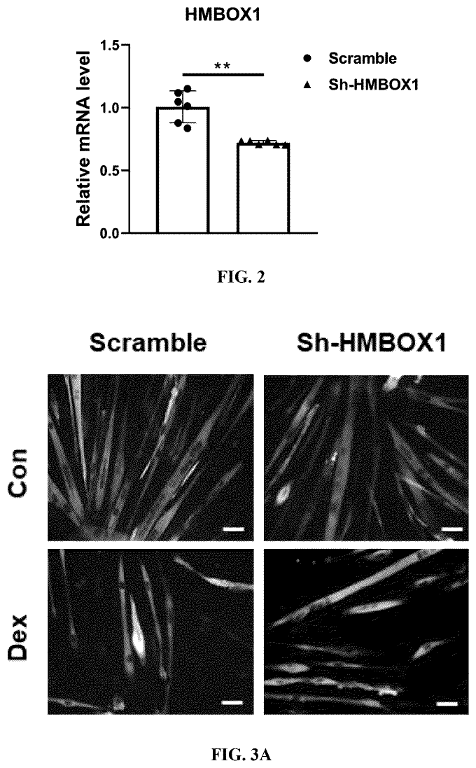

Simultaneously, the method for packaging Sh-HMBOX1 lentivirus was referenced to package the control lentivirus with the addition of scramble plasmid. The packaged Sh-HMBOX1 lentivirus and control lentivirus were transfected into differentiated myotube cells, with a transfection duration of 72 hours and a transfection titer of 1×10⁸ TU/mL. After the transfection, quantitative real-time polymerase chain reaction (PCR) was employed to detect the expression level of HMBOX1, with results shown in FIG. 2.

The results in FIG. 2 indicate that, compared to the HMBOX1 expression levels in the cells treated with the scramble control lentivirus, the HMBOX1 expression was significantly reduced in the cells treated with the packaged Sh-HMBOX1 lentivirus.

1.3 The constructed Sh-HMBOX1 plasmid was utilized to package lentivirus with the assistance of psPAX2 and PMD2.G vectors, achieving the goal of inhibiting HMBOX1. After 48 hours, a muscle atrophy model induced by Dex at the cellular level was established. The specific procedures were as follows:

### (1) C2C12 Myoblast Differentiation Culture:

The cells were cultured in high-glucose DMEM containing 10% FBS. When the cells reached approximately 70% confluence, the medium was replaced with differentiation medium containing 2% horse serum. Differentiation was allowed for 4 to 5 days until mature myotubes were observed, resulting in differentiated myotube cells.

### (2) Experimental Grouping:

Transfection with scramble control lentivirus+control for muscle atrophy model (Scramble+Con): Differentiated myotube cells were transfected with Scramble control lentivirus for 72 hours, with a transfection titer of 1×10⁸ transducing unit (TU)/mL.

Transfection with scramble control virus+Dex-induced muscle atrophy model (Scramble+Dex): Differentiated myotube cells were transfected with Scramble control lentivirus for 72 hours, with a transfection titer of 1×10⁸ TU/mL. After 48 hours of transfection, 50 μM Dex was added to induce muscle atrophy, and the experiment was concluded 24 hours post-Dex treatment.

Transfection with Sh-HMBOX1 Lentivirus+Control for muscle atrophy model (Sh-HMBOX1+Con): Differentiated myotube cells were transfected with Sh-HMBOX1 lentivirus for 72 hours, with a transfection titer of 1×10⁸ TU/mL.

Transfection with Sh-HMBOX1 Lentivirus+Dex-induced muscle atrophy model (Sh-HMBOX1+Dex): Differentiated myotube cells were transfected with Sh-HMBOX1 lentivirus for 72 hours, with a transfection titer of 1×10⁸ TU/mL. After 48 hours of transfection, 50 μM Dex was added to induce muscle atrophy, and the experiment was terminated 24 hours post-Dex treatment.

(3) After the experiment, immunofluorescence staining was performed using the specific antibody MF-20 (DSHB) to label muscle fibers, whose diameters were then measured. Subsequently, RNA was extracted from the muscle fiber cells and quantitative PCR was performed to assess the expression changes of muscle atrophy-specific genes Atrogin-1 and MuRF-1. This was done to determine whether the inhibition of HMBOX1 expression could suppress dexamethasone-induced muscle atrophy at the cellular level.

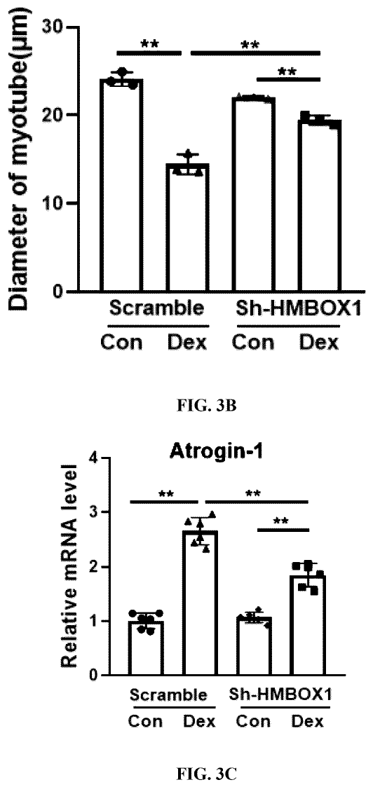

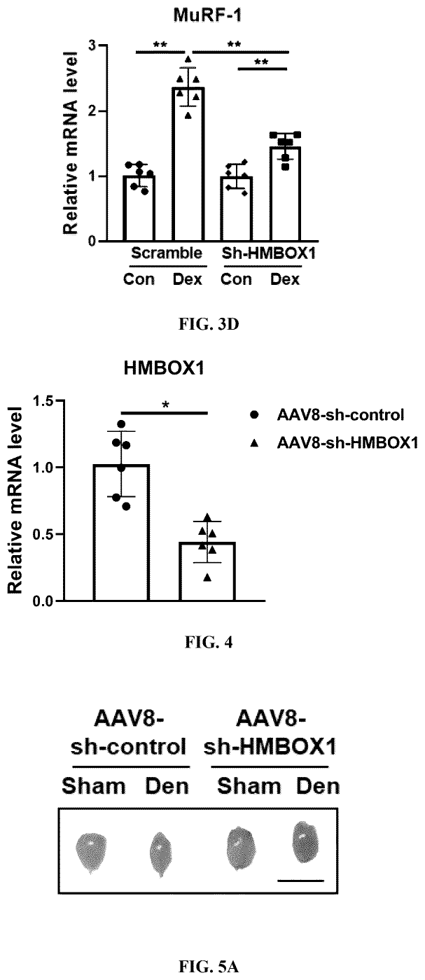

Results presented in FIGS. 3A-3D indicate that inhibiting HMBOX1 expression significantly increased the diameter of muscle fiber cells and markedly suppressed the elevation of muscle atrophy-specific ubiquitin ligases (such as Atrogin-1 and MuRF-1). These findings suggest that the suppression of HMBOX1 expression may inhibit the occurrence of dexamethasone-induced muscle atrophy at the cellular level.

### Example 2. Intervention with AAV8-Sh-HMBOX1 Adeno-Associated Virus in Mice Induced by Denervation-Induced Muscle Atrophy

### (1) Adeno-Associated Virus (AAV) Packaging:

The Sh-HMBOX1 plasmid constructed in Example 1 was utilized to package the virus into adeno-associated virus with the assistance of Helper and AAV shuttle vector systems. Specifically, 10 μg of AAV8 virus packaging plasmid (AAV Construct, Helper, serotype 8, PEI MAX) was transfected into 293T cells. After 10 hours, the complete culture medium was replaced, and the AAV8 virus was collected after 48 hours. The virus in the medium was concentrated by precipitation with 40% PEG-8000, stirred at 4° C. for 1 hour, and allowed to settle overnight. The virus precipitate was then centrifuged at 4,000 rpm for 30 minutes at 4° C. To resuspend the virus pellet, 5 mL of cell lysis buffer (lysis buffer: 150 mM NaCl, 20 mM Tris pH 8.0) was added. The virus was further concentrated by repeated freeze-thaw cycles. To the two virus solutions, 1 mM MgCl₂ and 250 U/mL Benzonase nuclease were added, mixed, and incubated at 37° C. for 45 minutes. The supernatant was collected after centrifugation at 4,000 rpm for 30 minutes at 4° C. Density gradient centrifugation was performed using 60%, 40%, 25%, and 17% iodixanol to purify the virus and afford the deno-associated virus AAV8-sh-HMBOX1, followed by high-speed centrifugation at 60,000 rpm for 2 hours at 4° C. to concentrate the virus. The viral titer was determined using quantitative real time polymerase chain reaction (qRT-PCR). Concurrently, the control adeno-associated virus AAV8-sh-control was constructed using the same method with the addition of the control plasmid. The packaged AAV8-sh-HMBOX1 and control viruses were directly injected into the gastrocnemius muscle of mice at a titer of 5×10¹¹ genome copies (GC)/mouse. Four weeks later, the gastrocnemius muscle tissue was collected, and the expression level of HMBOX1 was detected using qRT-PCR, with results shown in FIG. 4.

The results in FIG. 4 indicate that compared to the HMBOX1 expression levels in the gastrocnemius muscle after injection of AAV8-sh-control, the injection of AAV8-sh-HMBOX1 significantly reduced HMBOX1 expression in the gastrocnemius muscle.

### (2) Construction of Denervation-Induced Muscle Atrophy Model:

The viral titer was calculated, and the virus was directly injected into the gastrocnemius muscle at a titer of 5×10¹¹ GC/mouse. Three weeks later, the denervation (Den)-induced muscle atrophy model was constructed. The specific groups and procedures were as follows:

Injection with control adeno-associated virus+control for muscle atrophy model (AAV8-sh-control+Sham): Mice received an injection of control adeno-associated virus AAV8-sh-control into the gastrocnemius muscle at a titer of 5×10¹¹ GC/mouse, injected once. After three weeks, a sham surgery was performed, and the experiment was concluded one week post-surgery.

Injection with control adeno-associated virus+Den-induced muscle atrophy model group (AAV8-sh-control+Den): Mice received an injection of control adeno-associated virus AAV8-sh-control into the gastrocnemius muscle at a titer of 5×10¹¹ GC/mouse, injected once. After three weeks, a denervation surgery was performed, and the experiment was concluded one week post-surgery.

AAV8-sh-HMBOX1 adeno-associated virus group+control for muscle atrophy model (AAV8-sh-HMBOX1+Sham): Mice received an injection of adeno-associated virus AAV8-sh-HMBOX1 into the gastrocnemius muscle at a titer of 5×10¹¹ GC/mouse, injected once. After three weeks, a sham surgery was performed, and the experiment was concluded one week post-surgery.

Injection with AAV8-sh-HMBOX1 adeno-associated virus+Den-induced muscle atrophy model (AAV8-sh-HMBOX1+Den): Mice received an injection of AAV8-sh-HMBOX1 adeno-associated virus into the gastrocnemius muscle at a titer of 5×10¹¹ GC/mouse, injected once. After three weeks, a denervation surgery was performed, and the experiment was concluded one week post-surgery.

The denervation surgery involved using micro-scissors to cut the right sciatic nerve of C57BL/6 mice by 3-5 mm, ensuring that the nerve was completely severed. The sham surgery group underwent the same procedure without cutting the nerve.

(3) After the experiment, the gastrocnemius muscles of the mice were isolated. First, the weight changes of the gastrocnemius muscles were analyzed using an analytical balance. Next, embedded muscle samples for optical coherence tomography (OCT) were taken for cryosectioning, and wheat germ agglutinin (WGA) staining was conducted to statistically analyze changes in muscle fiber size. Finally, RNA was extracted from the gastrocnemius muscle tissue, and quantitative PCR was performed to detect changes in the expression levels of muscle atrophy-specific genes Atrogin-1 and MuRF-1. This was done to determine whether the inhibition of HMBOX1 expression could prevent or treat denervation-induced muscle atrophy at the animal level.

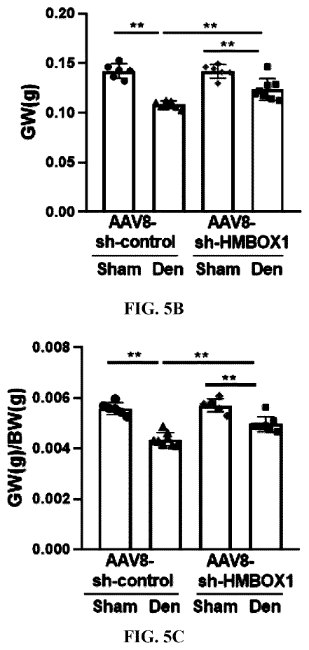

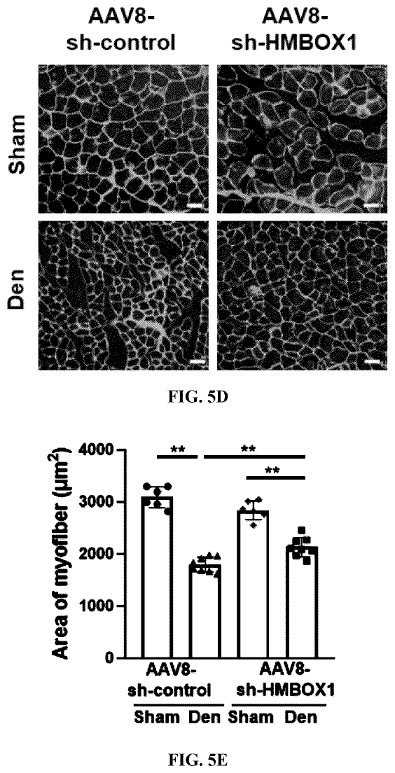

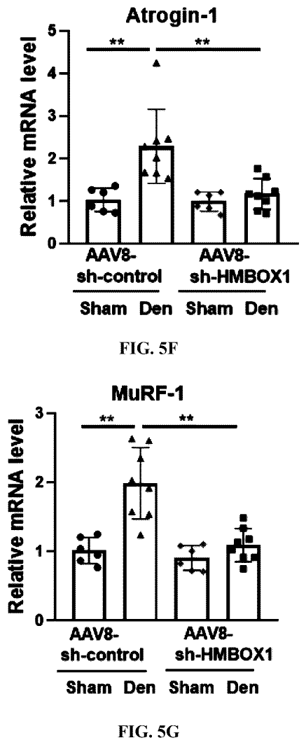

The results in FIGS. 5A-5G indicate that inhibiting HMBOX1 expression can slow down the reduction in muscle weight, mitigate the shrinkage of muscle fibers, and inhibit the increase of muscle atrophy-specific ubiquitin ligases (such as Atrogin-1 and MuRF-1). This suggests that the injection of AAV8-sh-HMBOX1 adeno-associated virus into the gastrocnemius muscle can effectively suppress the occurrence of denervation-induced muscle atrophy.

### (3) After the Experiment:

Immunofluorescence staining was performed using the specific antibody MF-20 (DSHB) to label the myotubes, and the diameter of the myotubes was quantified. Subsequently, RNA was extracted from the myotube cells, and quantitative PCR was conducted to detect changes in the expression levels of muscle atrophy-specific genes Atrogin-1 and MuRF-1. This was done to determine whether the inhibition of HMBOX1 expression could suppress the occurrence of Dex-induced muscle atrophy at the cellular level.

The results in FIGS. 3A-3B indicate that the inhibition of HMBOX1 expression significantly increased the diameter of myotube cells and markedly suppressed the elevation of muscle atrophy-specific ubiquitin ligases (such as Atrogin-1 and MuRF-1), suggesting that the inhibition of HMBOX1 expression can effectively prevent Dex-induced muscle atrophy at the cellular level.

### Example 3: Intervention with Adeno-Associated Virus AAV8-Sh-HMBOX1 after Imo-Induced Muscle Atrophy in Mice

In this example, the adeno-associated virus AAV8-sh-HMBOX1 and the AAV8-sh-control control adeno-associated virus prepared in Example 2 were utilized. A mouse model of Imo-induced muscle atrophy was constructed, and one week later, the adeno-associated virus AAV8-sh-HMBOX1 was directly injected into the gastrocnemius muscle at a titer of 1×10¹² GC/mouse. The specific groups and procedures were as follows:

Control for muscle atrophy model+injection with control adeno-associated virus (Sham+AAV8-sh-control): Mice underwent a sham surgery, and one week later, the gastrocnemius muscle was injected with the control adeno-associated virus AAV8-sh-control at a titer of 1×10¹² GC/mouse, injected once. The experiment concluded three weeks later.

Imo-induced muscle atrophy model+injection with control adeno-associated virus (Imo+AAV8-sh-control): Mice underwent Imo surgery, and one week later, the gastrocnemius muscle was injected with the control adeno-associated virus AAV8-sh-control at a titer of 1×10¹² GC/mouse, injected once. The experiment concluded three weeks later.

Control for muscle atrophy model control+Injection with adeno-associated virus AAV8-sh-HMBOX1 (Sham+AAV8-sh-HMBOX1): Mice underwent a sham surgery, and one week later, the gastrocnemius muscle was injected with the adeno-associated virus AAV8-sh-HMBOX1 at a titer of 1×10¹² GC/mouse, injected once. The experiment concluded three weeks later.

Imo-induced muscle atrophy model+injection with adeno-associated virus AAV8-sh-HMBOX1 (Imo+AAV8-sh-HMBOX1): Mice underwent Imo surgery, and one week later, the gastrocnemius muscle was injected with the AAV8-sh-HMBOX1 adeno-associated virus at a titer of 1×10¹² GC/mouse, injected once. The experiment concluded three weeks later.

(3) After the experiment, the gastrocnemius muscles of the mice were isolated. First, the weight changes of the gastrocnemius muscles were analyzed using an analytical balance. Next, embedded muscle samples for OCT were taken for cryosectioning, and WGA staining was conducted to statistically analyze changes in muscle fiber size. Finally, RNA was extracted from the gastrocnemius muscle tissue, and quantitative PCR was performed to detect changes in the expression levels of muscle atrophy-specific genes Atrogin-1 and MuRF-1. This was done to determine whether the inhibition of HMBOX1 expression could treat Imo-induced muscle atrophy at the animal level.

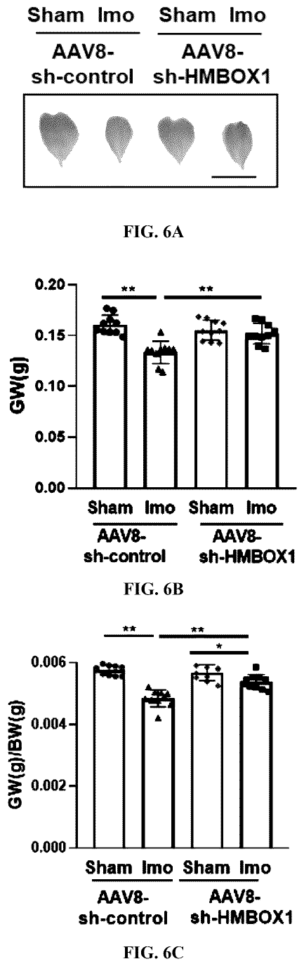

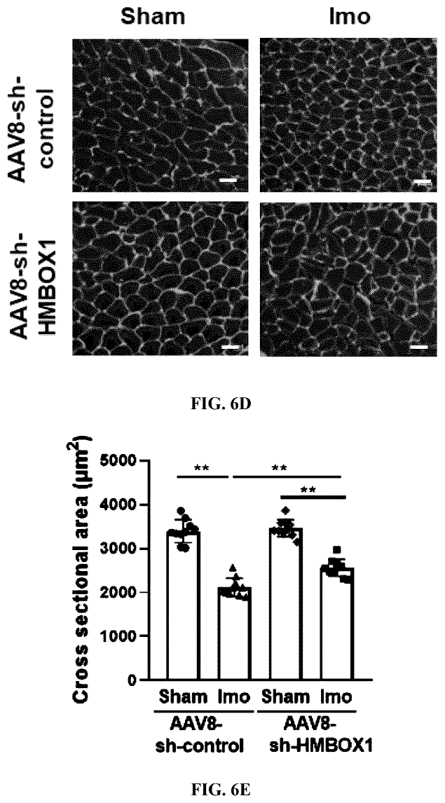

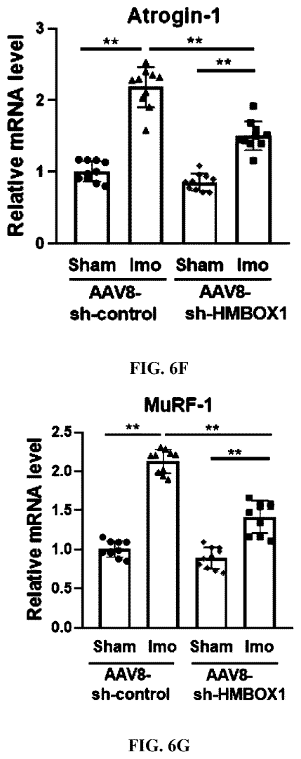

The results in FIGS. 6A-6G indicate that inhibiting HMBOX1 expression may slow down the reduction in muscle weight, mitigate the shrinkage of muscle fibers, and inhibit the increase of muscle atrophy-specific ubiquitin ligases (such as Atrogin-1 and MuRF-1). This suggests that the inhibition of HMBOX1 expression may effectively treat Imo-induced muscle atrophy.

The above description represents preferred embodiments of the present disclosure. It should be noted that those skilled in the art may make various modifications and refinements without departing from the principles of the disclosure. These modifications and refinements should also be considered within the protection scope of the present disclosure.

What is claimed is:

1. A method for preventing and/or treating muscle atrophy, comprising administering a homeobox containing 1 (HMBOX1) inhibitor to a subject in need thereof.

2. The method of claim 1, wherein the muscle atrophy comprises one or more of neurogenic muscle atrophy, disuse muscle atrophy, and steroid-induced muscle atrophy.

3. The method of claim 1, wherein the HMBOX1 inhibitor comprises one or more of a first regulator, a protease or a nuclease, and a second regulator; wherein the first regulator reduces HMBOX1 expression, the protease or the nuclease degrades an HMBOX1 product, and the second regulator decreases the HMBOX1 product.

4. The method of claim 3, wherein the first regulator comprises a small interfering RNA (siRNA), a short hairpin RNA (shRNA), or a mircroRNA (miRNA).

5. The method of claim 3, wherein the second regulator comprises an HMBOX1 antibody.

6. The method of claim 4, wherein the siRNA has the nucleotide sequence of SEQ ID NO: 1; and the shRNA has the target sequence of SEQ ID NO: 1.

7. The method of claim 1, wherein the HMBOX1 inhibitor slows down a reduction in muscle weight and a shrinkage in muscle fiber.

8. The method of claim 7, wherein the muscle atrophy comprises one or more of neurogenic muscle atrophy, disuse muscle atrophy, and steroid-induced muscle atrophy.

9. The method of claim 7, wherein the HMBOX1 inhibitor comprises one or more of a first regulator, a protease or a nuclease, and a second regulator; wherein the first regulator reduces HMBOX1 expression, the protease or the nuclease degrades an HMBOX1 product, and the second regulator decreases the HMBOX1 product.

10. The method of claim 9, wherein the first regulator comprises an siRNA, an shRNA, or a miRNA.

11. The method of claim 9, wherein the second regulator comprises an HMBOX1 antibody.

12. The method of claim 10, wherein the siRNA has the nucleotide sequence of SEQ ID NO: 1; and the shRNA has the target sequence of SEQ ID NO: 1.

13. The method of claim 1, wherein the HMBOX1 inhibitor significantly inhibits an increase in muscle atrophy-specific ubiquitin ligase.

14. The method of claim 13, wherein the muscle atrophy comprises one or more of neurogenic muscle atrophy, disuse muscle atrophy, and steroid-induced muscle atrophy.

15. The method of claim 13, wherein the HMBOX1 inhibitor comprises one or more of a first regulator, a protease or a nuclease, and a second regulator; wherein the first regulator reduces HMBOX1 expression, the protease or the nuclease degrades an HMBOX1 product, and the second regulator decreases the HMBOX1 product.

16. The method of claim 15, wherein the first regulator comprises an siRNA, an shRNA, or a miRNA.

17. The method of claim 15, wherein the second regulator comprises an HMBOX1 antibody.

18. A drug for prevention and/or treatment of muscle atrophy, comprising an active ingredient and a pharmaceutically acceptable carrier, wherein the active ingredient is the shRNA in the method of claim 4.

19. The drug of claim 18, wherein the siRNA has the nucleotide sequence of SEQ ID NO: 1; and the shRNA has the target sequence of SEQ ID NO: 1.

20. The drug of claim 18, wherein the muscle atrophy comprises one or more of neurogenic muscle atrophy, disuse muscle atrophy, and steroid-induced muscle atrophy.


SEQUENCE LISTING (4 sequences)

SEQ ID NO: 1 - DNA, 21 bp, synthetic construct
  source 1..21: mol_type = other DNA; note = siRNA for HMBOX1
gctggaaaccatgtctcacta

SEQ ID NO: 2 - DNA, 58 bp, synthetic construct
  source 1..58: mol_type = other DNA; note = Upstream primer for constructing Sh - HMBOX1 plasmid
ccgggctggaaaccatgtctcactactcgagtagtgagacatggtttccagctttttg

SEQ ID NO: 3 - DNA, 58 bp, synthetic construct
  source 1..58: mol_type = other DNA; note = Downstream primer for constructing Sh-HMBOX1 plasmid
aattcaaaaagctggaaaccatgtctcactactcgagtagtgagacatggtttccagc

SEQ ID NO: 4 - DNA, 1263 bp, synthetic construct
  source 1..1263: mol_type = other DNA; note = Coding sequence (CDS) of HMBOX1
atgctcagctcctttccagtggttttgctggaaaccatgtctcactacacagatgaacccagatttaccatagaacagatagacctgctccagcgtcttcggcgtaccgggatgaccaaacatgaaatccttcatgcattagaaactttggaccgtcttgatcaagagcatagtgataaatttggaaggaggtccagctacgggggaagctcatatgggaacagtaccaacaacgttccagcatcttcctctacagccacagcttccacgcagacccagcactcgggaatgtccccatcacccagcaacagttacgatacctccccactgccttgcactaccaatcaaaatgggagggagaacaatgatcgattgtccacatccaatgggaagatgtcaccatctcgctaccatgcaaacagcatgggtcagaggtcatatagctttgaggcctcagaagaggacctagatgtagatgataaagtggaggaattaatgaggagggacagcagtgtgataaaagaggaaatcaaagcctttcttgccaatcggaggatttcccaagcagttgttgcacaggtaacaggaatcagtcagagtcgaatctctcactggctgctgcagcagggatcagatctgagtgagcagaagaaaagggcgttctaccgatggtatcaacttgagaagacaaaccctggggctacgctaagtatgagacctgcccccattccaatagaggaccctgaatggagacaaacacctcccccagtctccgccacacctggaaccttccggcttcgacgagggagtagatttacctggagaaaggagtgcctagctgtcatggaaagttacttcaatgagaaccagtacccagatgaagcaaagagagaagaaattgccaatgcttgcaatgcagtcatacagaagccaggcaaaaagctgtctgacctggaacgagttacctctctgaaagtatataattggtttgctaatcgacggaaggagatcaagagaagagccaatatcgaagcagcaatcctggagagtcatgggatagatgtacagagtccaggaggccattccaacagtgacgatgtggacgggaacgactactccgagcaggatgacagcaccagccatagtgaccaccaagatcccatctcgctagctgtggagatggcggccgtcaaccacactatcttggcattggcccggcagggagccaatgaaatcaagacagaggccctggatgatgactga